(19) United States
(12) Patent Application Publication
(10) Pub. No.: US 2017/0119835 A1
(43) Pub. Date: May 4, 2017

(54) METHOD OF MAKING AN ADJUNCT TO POTENTIATE BLOCKING OF RIBOSOMAL FUNCTIONS IN TUMOR CELLS AND PREVENT BODY WEIGHT LOSS DURING CYCLOPHOSPHAMIDE CANCER THERAPY

(71) Applicants: Deepa Chitre, Warje (IN); Debendranath Dey, Warje (IN); Sunetra Chaskar, Wajre Malwadi (IN)

(72) Inventors: Deepa Chitre, Warje (IN); Debendranath Dey, Warje (IN); Sunetra Chaskar, Wajre Malwadi (IN)

(21) Appl. No.: 15/257,912

(22) Filed: Sep. 7, 2016

Related U.S. Application Data
(63) Continuation-in-part of application No. 13/322,150, filed on Nov. 22, 2011.

(30) Foreign Application Priority Data
May 22, 2009 (IN) 1283/MUM/2009

(51) Int. Cl. A61K 36/81; A61K 9/00; A61K 31/58

(52) U.S. Cl. CPC A61K 36/81; A61K 31/58; A61K 9/0053; A61K 2236/333

(57) ABSTRACT

A method of obtaining one or more fractions from a plant material of Withania somnifera (WS) is disclosed. The method includes subjecting the plant material to hydro-alcoholic extraction in presence of a water-insoluble solvent to obtain at least one extract. The method further includes subjecting the at least one extract obtained from the hydro-alcoholic extraction to at least one of de-pigmentation, de-fatting and detoxification process to obtain the one or more fractions. The one or more fractions thus obtained contain Withaferin A in a concentration greater than concentrations of other pharmacologically active ingredients present in the one or more fractions. The one or more fraction thus obtained and one or more compositions containing the one or more fractions are effective in inhibiting proliferation of mammalian cancerous cells.

### RELATED APPLICATIONS

This patent application claims the benefit of priority to U.S. patent application Ser. No. 13/322,150 filed Nov. 22, 2011, a National Phase application of PCT Patent Application No. PCT/IN2010/000348, filed May 21, 2010, which claims the benefit of priority to India Patent Application No. 1283/MUM/2009, filed May 22, 2009, all of these incorporated by reference herein, in their entirety.

### FIELD OF THE INVENTION

The invention generally relates to cancer chemotherapies, including potentiating tumor-reducing action of chemotherapy agents, and reducing undesirable side effects of chemotherapy agents, such as cyclophosphamide.

### BACKGROUND

The conventional treatments of various cancers usually include chemotherapy alone or in combination with radiotherapy. Chemotherapy using synthetic anti-cancer drugs alone or in combination with radiotherapy is known to cause several serious and unpleasant side effects like loss of hair, nausea, vomiting, weakness and fall in blood counts leading to life threatening infections, hemorrhages and respiratory distress.

Therefore, it is desirable to design treatments that are associated with fewer side-effects and lesser toxicity as compared with the conventional chemotherapy alone or in combination with radiotherapy. On the other hand, medicinal herbs and medicines obtained therefrom are known to be associated with minimal side-effects as compared to the conventional treatments options including synthetic drugs. Therefore, use of various herbs in treatment of cancer has been widely studied in the art. Withania somnifera (WS) is one such herb widely studied for its effectiveness in various diseases including cancer. Different pharmacologically active ingredients present in WS impart various medicinal qualities useful in treatment of a number of human ailments and diseases, including cancers.

The pharmacologically active ingredients present in WS, especially Withaferins, have been found effective in various cancers. Studies have shown that Withaferins, especially, Withaferin A, is very useful in the treatment of cancer. Further, the pharmacologically active ingredients present in WS have also been found to reduce side-effects associated with the conventional chemotherapy and/or radiotherapy. Therefore, efforts have been made to develop methods to extract Withaferins from WS. The current methods of extracting Withaferins, for example, Withaferin A, are focused on extraction of pure Withaferins. However, pure Withaferins, especially Withaferin A, are found to be associated with acute cytotoxicity.

There is therefore a need for an improved method for extraction of one or more fractions, containing Withaferin A along with other pharmacologically active ingredients that are effective in various cancers and are associated with minimal cytotoxicity.

### BRIEF DESCRIPTION OF THE DRAWINGS

Certain embodiments of the disclosure will hereafter be described with reference to the accompanying drawings, wherein like reference numerals denote like elements. It should be understood, however, that the accompanying figures illustrate the various implementations described herein and are not meant to limit the scope of various technologies described herein.

The accompanying figures, incorporated in and form part of the specification, serve to illustrate various examples in accordance with the present invention.

FIG. 1A illustrates a chromatograph depicting the HPLC profile of the fraction (BV-3115) in accordance with Example 2.

FIG. 1B illustrates a table depicting the peak values of various pharmacologically active ingredients present in the fraction (BV-3115) obtained during HPLC analysis of the fraction in accordance with Example 2.

FIG. 2 illustrates effect of the fraction (BV-3115) on inhibiting Liver Cancer Cell Line (Hep G2 Cells) using Sulforhodamine B (SRB) Assay.

FIG. 3 illustrates effect of BV-3115 on inhibiting proliferation of Human Prostate Cancer Cell Line (PC-3 Cells).

FIG. 4 illustrates effect of BV-3115 in inhibiting proliferation of Human Prostate Cancer Cell Line (DU-145 Cells), in-vitro.

FIG. 5 illustrates effect of BV-3115 in inhibiting proliferation of Human Breast Cancer Cell Line (MCF-7 Cells), in-vitro.

FIG. 6 illustrates effect of BV-3115 in inhibiting proliferation of Human Colon Cancer Cell Line (Colo 320 DM Cells), in-vitro.

FIG. 7 illustrates body weight gain/loss in the animals used in Sarcoma Model as compared with the control group in accordance with an exemplary embodiment of the invention.

FIG. 8 illustrates percent inhibition of rat paw edema using BV-3115 as compared with Diclofenac and control.

FIG. 9 illustrates an HPLC chromatogram of an example BV-3115 compound.

FIG. 10 illustrates identification of HPLC chromatogram peaks of the example BV-3115 compound of FIG. 9.

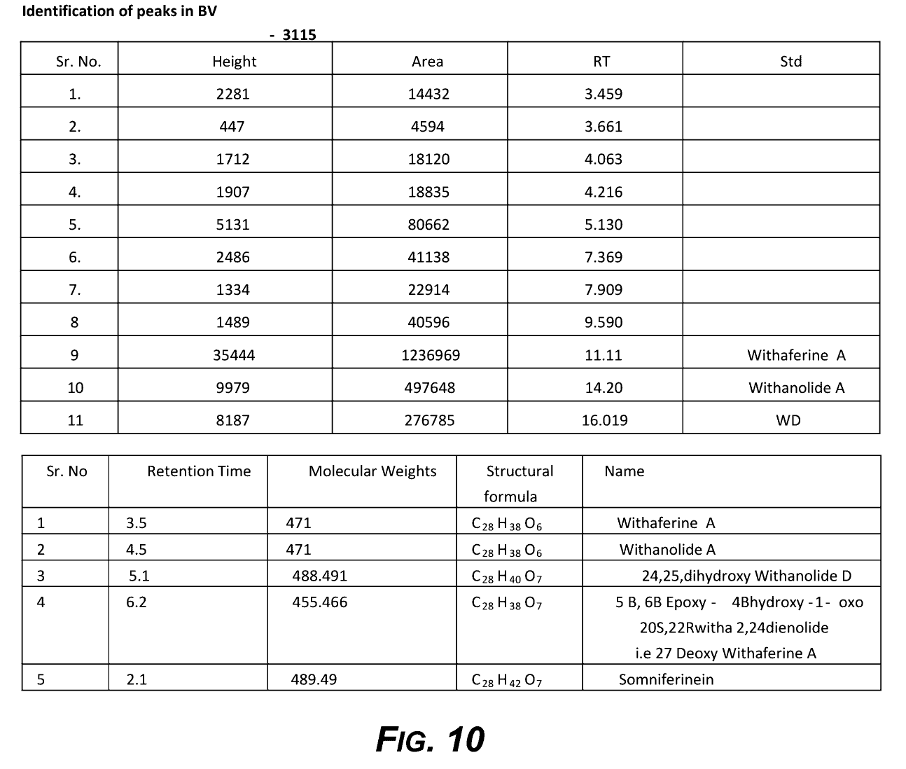

FIG. 11 illustrates a liquid chromatography-mass spectrometry (LCMS) analysis of the example BV-3115 compound.

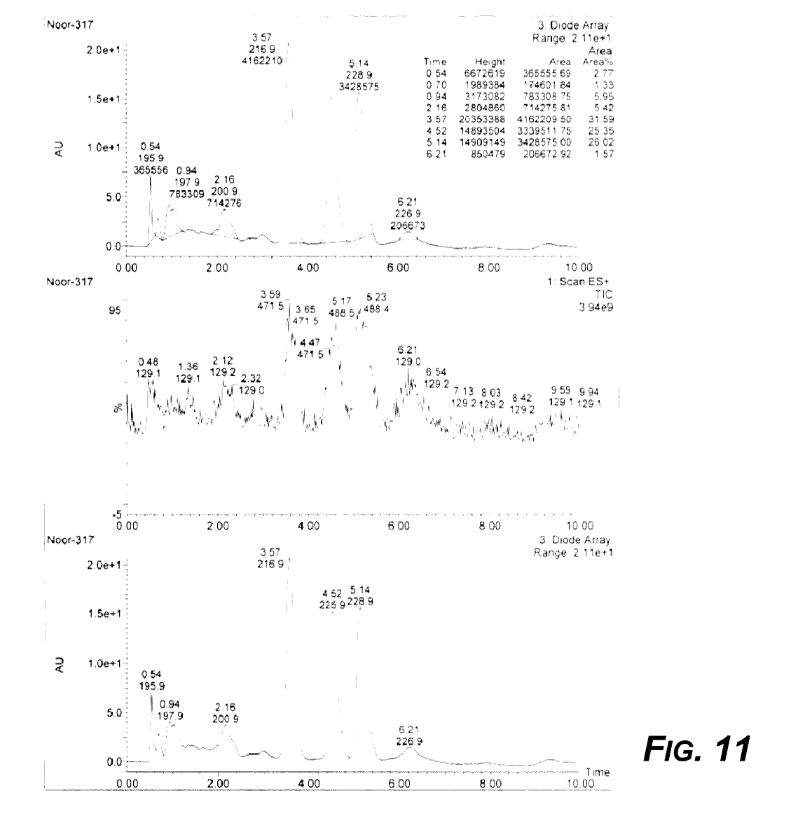

FIG. 12 illustrates a liquid chromatography-mass spectrometry (LCMS) analysis of the example BV-3115 compound.

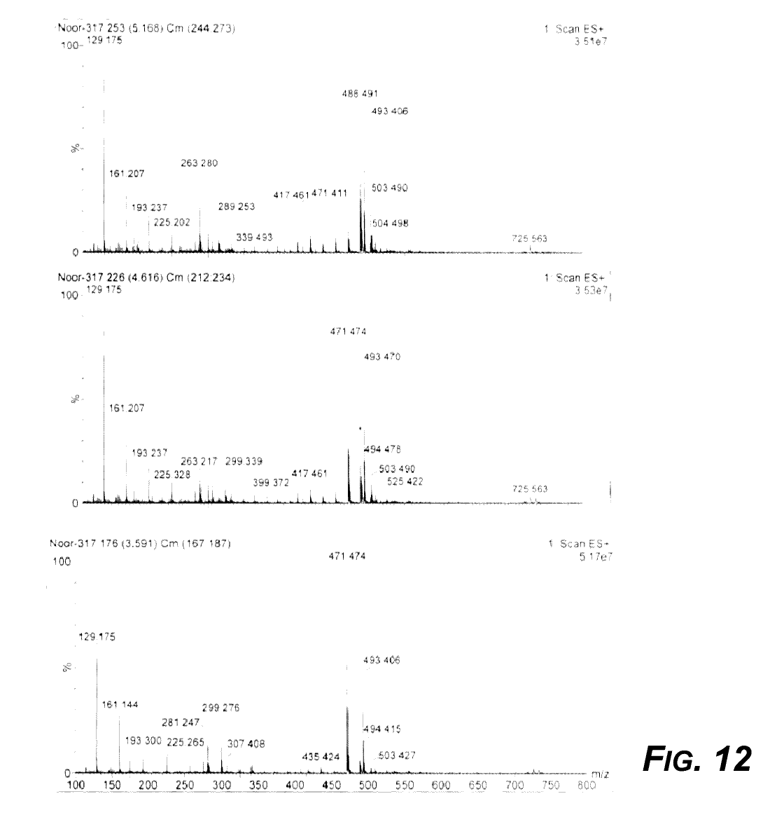

FIG. 13 illustrates a body weight change study comparing administration of example compounds.

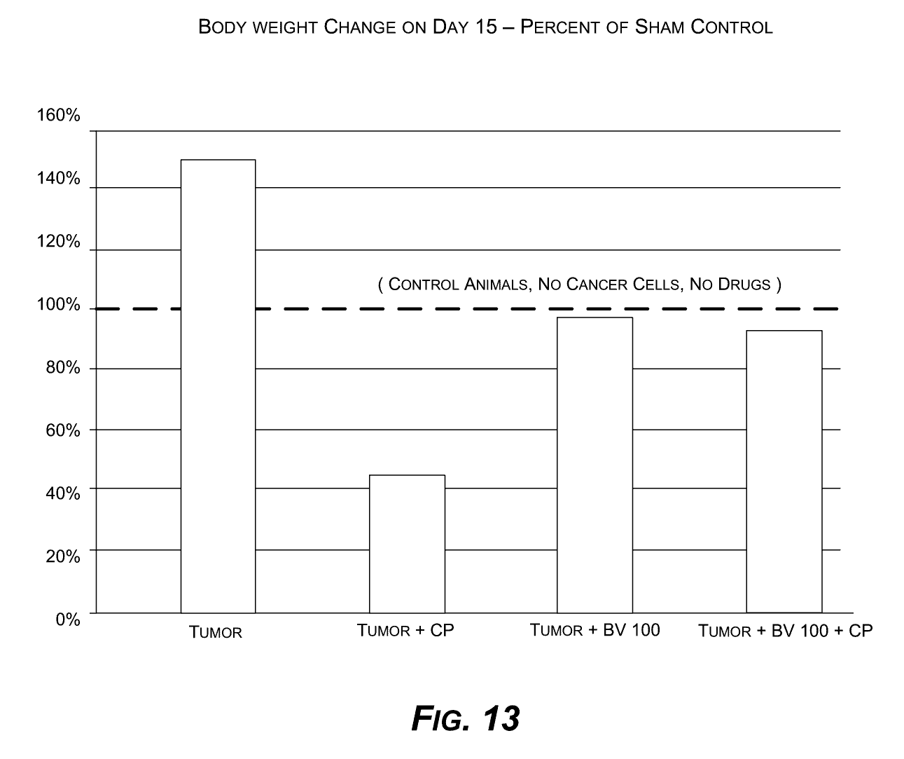

FIG. 14 illustrates changes in ribosomal function of cancer cells with different doses of the example BV-3115 compound.

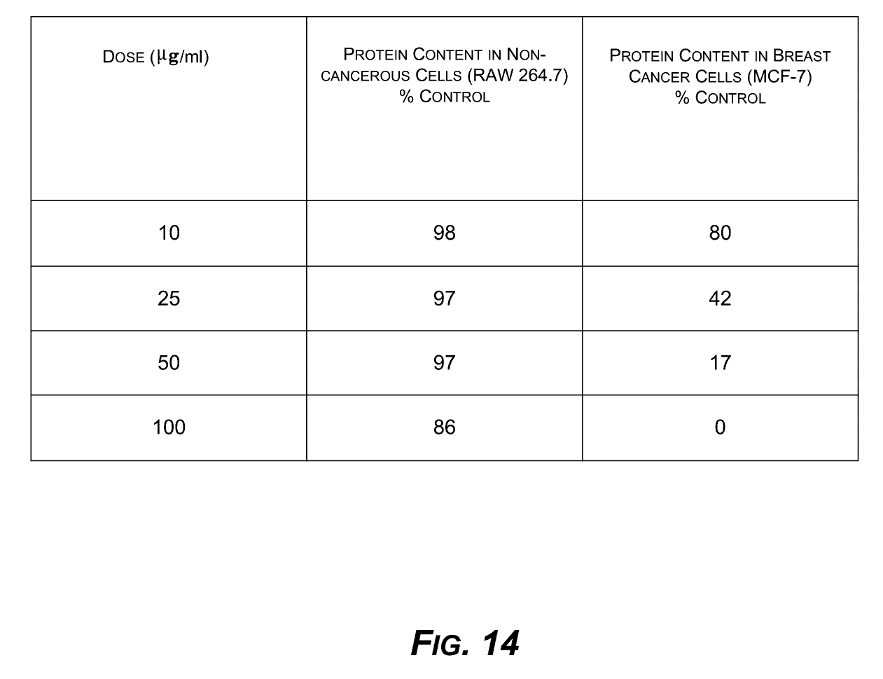

### DESCRIPTION

This disclosure describes methods of making an adjunct to potentiate blocking of ribosomal functions in tumor cells and prevent body weight loss during cyclophosphamide cancer therapy. Before describing in detail, the embodiments that are in accordance with the present invention, it should be observed that the embodiments reside primarily in combinations of method steps and constituents related to method of extracting one or more fractions from Withania somnifera (WS), the one or more fractions obtained from WS and compositions containing the one or more fractions. Accordingly, the method steps, the one or more fractions and the compositions containing the one or more fractions have been described, showing only those specific details that are pertinent to understanding the embodiments of the present invention so as not to obscure the disclosure with details that will be readily apparent to those of ordinary skill in the art.

In this document, terms such as “comprises,” “comprising,” or any other variation thereof, are intended to cover a non-exclusive inclusion, such that a process and method, comprises a list of steps does not include only those steps but may include other steps not expressly listed or inherent to such process and method.

Generally speaking, pursuant to various embodiments, the invention provides methods for obtaining one or more fractions containing Withaferin A in a concentration predominantly greater than concentrations of other pharmacologically active ingredients present in the one or more fractions from a plant material obtained from WS. WS is a shrub belonging to family Solanaceae or nightshade and grows predominantly in India, Nepal, Pakistan, Sri Lanka and Bangladesh. WS is also known as Ashwagandha, Indian ginseng, Winter cherry, Ajagandha, and Kanaje.

In accordance with various embodiments, the method includes extracting the one or more fractions from the plant material obtained from WS. The plant material may be one or more of one or more fresh parts of WS and one or more dried parts of WS. The plant material may further be one or more of, but are not limited to, whole plant of WS, leaves, roots, bark, stems, flowers, fruits, exudates, and any other part of WS containing one or more pharmacologically active ingredients.

The plant material may be processed chemically or physically before initiating the method. Physical processing of the plant material may include, for example, but not limited to, size reduction. Whereas, chemical processing of the plant material may include, for example, but not limited to, treating the plant material with one or more chemicals, washing with water, and the like. Optionally, an un-processed plant material may also be used in the method. In an exemplary embodiment, the plant material is a coarse powder of dried roots of WS.

The plant material thus obtained from WS is then subjected to hydro-alcoholic extraction in presence of a water-insoluble solvent. The hydro-alcoholic extraction includes soaking the plant material in a mixture of aqueous alcohol and the water-insoluble solvent for a predetermined time. The water-insoluble solvent used in the hydro-alcoholic extraction may be one of, but are not limited to, chloroform, acetone, dichloromethane and tetra-chloromethane. Any other similar water-insoluble solvent may be used in the hydro-alcoholic extraction without departing from the scope of the invention. In an exemplary embodiment, the water-insoluble solvent is chloroform.

An alcohol present in the aqueous alcohol may be, for example, but is not limited to, methanol, ethanol, propanol, amyl alcohol, isopropyl alcohol and any other alcohol with a polarity similar to polarity of the alcohol. In an exemplary embodiment, the alcohol present in the aqueous alcohol is methanol. Further, the concentration of alcohol in the aqueous alcohol may range from 6% v/v to 95% v/v. The concentration of alcohol may be selected based on the plant material used in the method. For example, when the plant material is the fresh plant material, the concentration of the alcohol in the aqueous alcohol may range from 6% v/v to 90% v/v. Whereas, when the plant material is the dry plant material, the concentration of the alcohol in the aqueous alcohol may range from 20% v/v to 86% v/v. More preferably, the concentration of the alcohol in the aqueous alcohol may range from 60% v/v to 80% v/v irrespective of whether the plant material is the dry plant material or the fresh plant material. In an exemplary embodiment, the concentration of the alcohol in the aqueous alcohol is 60% v/v. However, the concentration of the alcohol in the aqueous alcohol may be varied based on the plant material used in the method without departing from the scope of the invention.

The plant material is soaked in the mixture of the aqueous alcohol and the water-insoluble solvent for a predetermined time. In an embodiment, the predetermined time may range from 6 hours to 48 hours. For example, the plant material may be soaked in the mixture of the aqueous alcohol and the water-insoluble solvent overnight (8 hours to 12 hours). In another embodiment, the predetermined time of soaking the plant material may range from few hours to several days depending upon the conditions and objectives of the hydro-alcoholic extraction without departing from the scope of the invention.

The mixture may be occasionally stirred while the mixture is allowed to soak. The stirring may be accomplished using methods known in the art. For example, the stirring may be achieved by using any appropriate laboratory or industrial stirrer/shaker or optionally the mixture may be stirred manually by using an appropriate stirrer, for example, a rod.

Soaking of the plant material in the mixture for the predetermined time constitutes maceration. Generally, the secondary metabolites that constitute the pharmacologically active ingredients in the plant material are located deep inside tissues of the plant material. Therefore, in order to extract the pharmacologically active ingredients effectively, the water-insoluble solvent and the aqueous alcohol present in the mixture should penetrate the tissues and cells of the plant material so that the secondary metabolites dissolve in the solvent and may be further extracted.

Water present in the aqueous alcohol swells the tissues of the plant material and dissolves one or more pharmacologically active ingredients of WS. Further, the alcohol being a more potent extraction solvent with a wide range of solubility dissolves additional pharmacologically active ingredients present in WS. The use of water-insoluble solvent helps in extraction of other pharmacologically active ingredients of WS in addition to the pharmacologically active ingredients extracted by the alcohol and water. Thus, soaking of the plant material in the mixture of the aqueous alcohol and the water-insoluble solvent results in extraction of the pharmacologically active ingredients of WS in two different phases. The two different phases may be a hydro-alcoholic phase and a water-insoluble phase.

After the predetermined time is over, the mixture is filtered. The filtration may be carried out using the methods generally known and used in the art of liquid-liquid extraction. Alternatively, the filtration may be achieved by using appropriate laboratory or industrial filtration procedures. As a result of filtering the mixture, a first residue and a first filtrate is obtained.

The first residue obtained as a result of filtering the mixture is kept aside and used later for re-extraction. Whereas, the first filtrate obtained as a result of filtering the mixture is allowed to settle. The first filtrate includes two immiscible layers. The two immiscible layers include a first aqueous layer and a first non-aqueous layer. The first aqueous layer and the first non-aqueous layer become visually distinct when the mixture is allowed to settle. The first aqueous layer and the first non-aqueous layer thus visually distinguished are then separated using one or more methods known in the art. For example, the first aqueous layer and the first non-aqueous layer may be separated using a separating funnel when the method is carried out on a laboratory scale. Whereas, the first aqueous layer and the first non-aqueous layer may be separated using appropriate solvent partitioning techniques known in the art of liquid-liquid extraction when the method is carried out on an industrial scale.

The first aqueous layer and the first non-aqueous layer thus separated are then separately subjected to a step of concentrating the first aqueous layer and the first non-aqueous layer to obtain a first dry extract and a second dry extract respectively. The step of concentrating the first aqueous layer and the first non-aqueous layer may include, one or more of, but are not limited to, drying, evaporating and vacuum evaporating the first aqueous layer and the first non-aqueous layer, separately.

For example, the first aqueous layer is subjected to evaporation using the methods known in the art. The evaporation may be carried out till the time the water and the alcohol in the first aqueous layer are completely evaporated so as to obtain the first dry extract. The first dry extract thus obtained constitutes a first hydro-alcoholic fraction. Whereas, the first non-aqueous layer is subjected to evaporation using the methods known in the art. The evaporation may be carried out till the time the water-insoluble solvent in the first non-aqueous layer is completely evaporated so as to obtain the second dry extract. The second dry extract thus obtained constitutes a first non-aqueous fraction. The first dry extract and the second dry extract are then weighed separately. The first dry extract is kept aside and the second dry extract is used for subsequent steps of the method.

The second dry extract is then subjected to one or more of a de-pigmentation process, a de-fatting process, and a detoxification process. The one or more of the de-pigmentation process, the de-fatting process, and the detoxification process may include treating or dissolving the second dry extract in one or more of a de-pigmenting agent, a de-fatting agent, and a detoxifying agent. For example, the second dry extract may be dissolved in the one or more of the de-pigmenting agent, the de-fatting agent, and the detoxifying agent. Use of the one or more of the de-pigmenting agent, the de-fatting agent, and the detoxifying agent as solvents in the method facilitates removal of one or more of lipids, pigments and toxins from the plant material which may otherwise impart toxicity to the one or more fractions obtained from the plant material.

The one or more of the de-pigmenting agent, the de-fatting agent, and the detoxifying agent may be a single organic solvent having one or more properties of de-pigmenting, de-fatting and detoxifying the second dry extract. Alternatively, the de-pigmenting agent, the de-fatting agent, and the detoxifying agent may include two or more agents having one or more properties of de-pigmenting, de-fatting and detoxifying the second dry extract. The one or more of the de-pigmenting agent, the de-fatting agent, and the detoxifying agent may be selected from the group consisting of pentane, hexane, heptanes, diethyl ether, petroleum ether, ethylene chloride, methylene chloride, cyclohexane, solvent ether and the like. In an exemplary embodiment, the process of de-pigmentation, de-fatting and detoxification includes dissolving the second dry extract in hexane.

Dissolution of the second dry extract yields a first solution. The first solution is then filtered to obtain a second residue and a second filtrate. The second filtrate thus obtained is discarded. Whereas the second residue obtained as a result of filtering the first solution is dried and weighed. The second residue thus obtained constitutes a first fraction of the one or more fractions. The first fraction contains Withaferin A in a concentration predominantly greater than concentrations of other pharmacologically active ingredients present in the first fraction.

Thereafter, the first residue obtained as a result of filtering the mixture and that is kept aside, earlier, is subjected to a re-extraction process. The re-extraction process includes subjecting the first residue to hydro-alcoholic extraction in presence of the water-insoluble solvent. The hydro-alcoholic extraction includes soaking the first in the mixture of aqueous alcohol and the water-insoluble solvent for a particular time. In an embodiment, the particular time may range from 6 hours to 48 hours. The mixture may be occasionally stirred while the mixture is allowed to soak. After the particular time is over, the mixture is filtered to obtain a third residue and a third filtrate.

The third filtrate thus obtained includes two immiscible layers. The two immiscible layers include a second aqueous layer and a second non-aqueous layer. The second aqueous layer and the second non-aqueous layer are then separated and concentrated separately to obtain a third dry extract and a fourth dry extract respectively. The fourth dry extract thus obtained constitutes a second non-aqueous fraction. The third dry extract and the fourth dry extract are then weighed separately. The third dry extract is kept aside and the fourth dry extract is used for subsequent steps of the re-extraction process.

Subsequently, the fourth dry extract is dissolved in the one or more of the de-pigmenting agent, the de-fatting agent, and the detoxifying agent. Dissolution of the fourth dry extract yields a second solution. The second solution is then filtered to obtain a fourth residue and a fourth filtrate. The fourth residue thus obtained constitutes a second fraction of the one or more fractions. The second fraction contains Withaferin A in a concentration predominantly greater than concentrations of other pharmacologically active ingredients present in the second fraction.

A total yield of the one or more fractions is calculated as sum of yields of the first fraction and the second fraction. The one or more fractions thus obtained contain Withaferin A in a concentration predominantly greater than the concentrations of other pharmacologically active ingredients present in the one or more fractions.

In an embodiment, the method of extracting one or more fractions from WS includes soaking dried root powder of WS in a mixture of 60% methanol and chloroform overnight (about 8 hours to 12 hours). Thereafter, the mixture is filtered to obtain a first residue and a first filtrate. The first residue is kept aside for subsequent use in a re-extraction process. The first filtrate is allowed to settle. The first filtered that is allowed to settle includes two immiscible layers. The two immiscible layers include a methanol layer and a chloroform layer. The methanol layer and the chloroform layer are separated using a separating funnel. Subsequently the methanol layer and the chloroform layer are separately subjected to evaporation to obtain a first dry aqueous methanolic extract and a first dry chloroform extract.

The first dry chloroform extract is then dissolved in hexane to obtain a first solution. Dissolution of the first dry chloroform extract in hexane allows one or more of de-fatting, de-pigmentation and detoxification of the plant material (dried root powder of WS) being extracted. The first solution is then filtered to obtain a second residue and a second filtrate. The second residue thus obtained constitutes a first fraction of the one or more fraction containing Withaferin A in a concentration greater than concentrations of other pharmacologically active ingredients present in the first fraction.

Thereafter, the first residue, obtained earlier, is subjected to the re-extraction process. The re-extraction process includes soaking the first residue in a fresh mixture of 60% methanol and chloroform overnight (about 8 hours to 12 hours). The mixture is then filtered to obtain a third residue and a third filtrate. The third residue may either be discarded or subjected to further re-extraction. The third filtrate is allowed to settle. The third filtrate that is allowed to settle includes two immiscible layers. The two immiscible layers include a methanol layer and a chloroform layer. The methanol layer and the chloroform layer are separated using a separating funnel. Subsequently the methanol layer and the chloroform layer are separately subjected to evaporation to obtain a second dry aqueous methanolic extract and a second dry chloroform extract.

The second dry aqueous methanolic extract constitutes a second aqueous methanolic fraction. Whereas the second dry chloroform extract is further dissolved in hexane to obtain a second solution. The second solution is then filtered to obtain a fourth residue and the fourth filtrate. The fourth filtrate is discarded. Whereas the fourth residue is dried and weighed separately. The fourth residue thus obtained constitutes a second fraction of the one or more fractions. The second fraction thus obtained contains Withaferin A in a concentration predominantly greater than concentrations of other pharmacologically active ingredients present in the second fraction. Total yield of the one or more fractions may be calculated as sum of yields of the first fraction and the second fraction.

The one or more fractions obtained in accordance with various embodiments contain Withaferin A in a concentration greater than concentrations of other pharmacologically active ingredients present in the one or more fractions. Further, the one or more fractions obtained in accordance with various embodiments are less toxic in mammals as compared to pure Withaferin A. The one or more fractions are effective in inhibiting proliferation of mammalian cancer cells. The mammalian cancer cells may be cells of one or more body organ of the mammal. The one or more body organ of the mammal may include, for example, but are not limited to, prostate, breast, colon, rectum, mouth, tongue, esophagus, stomach, pancreas, liver, spleen, brain, lung, bronchus, urinary bladder, cervix, ovaries, uterus, testes, thyroid, bone, cartilage, blood, lymphatic system, and skin. In other words, the one or more fractions obtained in accordance with various embodiments of the invention are effective in treatment of various cancers in mammals including human.

The one or more fractions, when used in combination with one or more of chemotherapy and radio therapy, are effective in reducing one or more side-effects associated with the one or more of chemotherapy and radio therapy. Also, the one or more fractions are effective in reducing toxicity otherwise associated with the one or more of chemotherapy and radio therapy.

Further, the one or more fractions are also effective in inhibiting one or more pro-inflammatory cytokines in mammals including human. The one or more pro-inflammatory cytokines may include for example, but are not limited to, Tumor Necrosis Factors (TNF), Interleukins (IL) and Cyclo-oxygenases (COX). The one or more fractions are effective in treating one or more inflammatory diseases. The one or more inflammatory diseases may be one or more of, but are not limited to, Arthritis, Ankylosing Spondylitis, Psoriasis, Rheumatoid Arthritis, Osteoarthritis, Multiple sclerosis, Atherosclerosis and Alzheimer's Disease. Additionally, the one or more fractions may inhibit one or more enzymes responsible for one or more diseases. The one or more enzymes may include for example, but not limited to, phosphodiesterase (PDE) Accordingly, the one or more fraction may be useful in the treatment of Asthma and other PDE mediated diseases.

Pursuant to various embodiments, the invention also provides a composition containing the one or more fractions obtained in accordance with various embodiments of the invention and one or more excipients. The one or more fractions present in the composition contain Withaferin A in a concentration greater than concentrations of other pharmacologically active ingredients present in the one or more fractions. The composition may be formulated as one of, for example, but not limited to, a tablet, a capsule, a suspension, a solution, an emulsion, and the like. Further, the composition may be formulated as any other dosage form suitable for delivering the one or more fractions to a subject without departing from the scope of the invention.

The composition is less toxic in mammals as compared to pure Withaferin A. The composition is effective in inhibiting proliferation of mammalian cancer cells. The mammalian cancer cells may be cells of one or more body organ of the mammal including, for example, but are not limited to, prostate, breast, colon, rectum, mouth, tongue, esophagus, stomach, pancreas, liver, spleen, brain, lung, bronchus, urinary bladder, cervix, ovaries, uterus, testes, thyroid, bone, cartilage, blood, lymphatic system, and skin. In other words, the composition in accordance with various embodiments of the invention is effective in treatment of various cancers in mammals including human.

The composition, when used in combination with one or more of chemotherapy and radio therapy, is effective in reducing one or more side-effects associated with the one or more of chemotherapy and radio therapy. Also, the composition is effective in reducing toxicity otherwise associated with the one or more of chemotherapy and radio therapy.

Further, the composition is also effective in inhibiting one or more pro-inflammatory cytokines in mammals including human. The one or more pro-inflammatory cytokines may include for example, but are not limited to, Tumor Necrosis Factors (TNF), Interleukins (IL) and Cyclo-oxygenases (COX). The composition is effective in treating one or more inflammatory diseases. The one or more inflammatory diseases may be one or more of, but are not limited to, Arthritis, Ankylosing Spondylitis, Psoriasis, Rheumatoid Arthritis, Osteoarthritis, Multiple sclerosis, Atherosclerosis and Alzheimer's Disease. Additionally, the composition may inhibit one or more enzymes responsible for one or more diseases. The one or more enzymes may include for example, but not limited to, phosphodiesterase (PDE). Accordingly, the composition may be useful in the treatment of Asthma and other PDE mediated diseases.

### Example 1

The dried roots of Withania somnifera (WS) were obtained from a local herb supplier in Pune, India. Methanol, Chloroform and Hexane were obtained from Merck, India. Water and all other reagents used were of analytical grade. The dried roots of WS were then coarsely powdered. The coarse powder of the matured roots of WS thus obtained (1 Kg) was transferred to a 10 L flask. 3 L methanol (60% v/v) was then added to the flask followed by addition of 4 L of chloroform. The resultant mixture was then allowed to soak overnight (8 hours to 12 hours). The mixture was intermittently stirred. After 8 hours to 12 hours the mixture was filtered to obtain the first residue and the first filtrate.

The first filtrate was allowed to settle. The first filtrate once settled had two immiscible layers. The two immiscible layers included an aqueous methanol layer and a chloroform layer. The chloroform layer was separated. Thereafter, the chloroform layer was then concentrated on a rotary evaporator under reduced pressure and dried at 50.degree. C. to obtain the chloroform soluble fraction (about 23 g). The chloroform soluble fraction thus obtained was then dissolved in hexane (2 L). The resultant solution was filtered to obtain the second residue and the second filtrate. The second residue was then dried at 50.degree. C. and stored as a first fraction. The first residue obtained as a result of filtering the mixture was then subjected to re-extraction by repeating the steps mentioned above to obtain a second fraction. The first fraction and the second fraction were mixed and stored as BV-3115.

The fraction, BV-3115, was then used for various studies to determine concentration of various constituents present in the fraction, therapeutic effectiveness of the fraction (BV-3115) in various cancers using animal studies, in-vivo and in-vitro studies, as disclosed in the examples below.

### Example 2

The fraction, BV-3115, obtained in accordance with Example 1 above was extracted with methanol and subjected to High Performance Liquid Chromatography (HPLC) analysis. HPLC Systems Waters M-32 (2487 dual .lamda. detector and 515 pump) was used for the HPLC analysis. Column width was set at 250 mm.times.4.6 mm (RP C18) with 5 .mu. packing. Methanol:Water in concentration ratio of 60:40 was used as mobile phase. Sample flow rate was set at 1 ml/min (Mode—isocratic) and run time was kept as 20 min. The analysis was carried out at 215 nm.

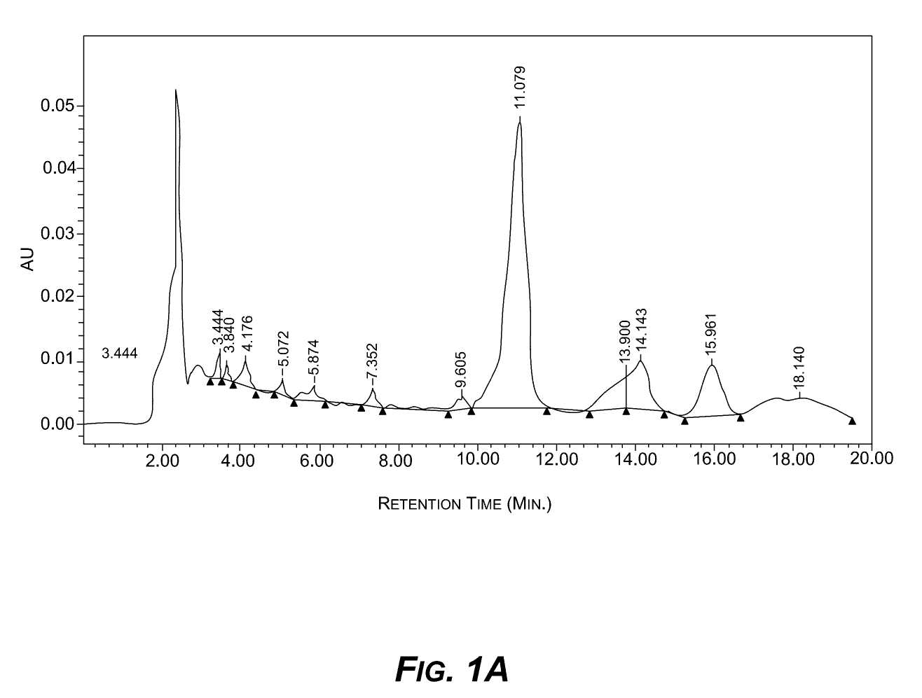

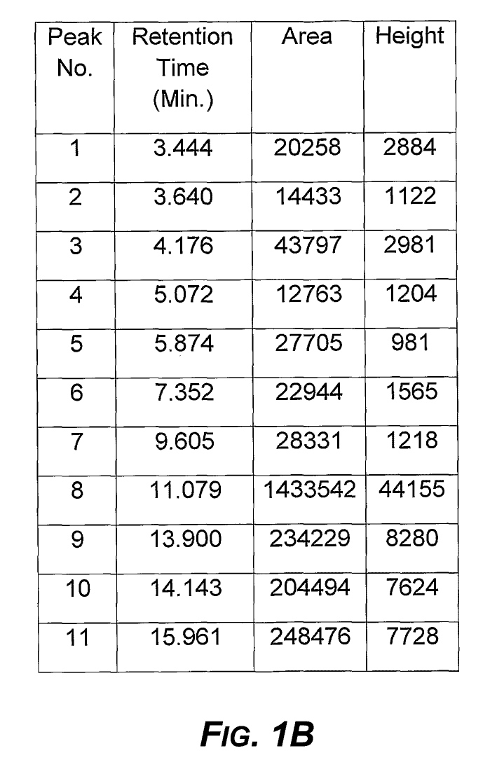

FIG. 1A illustrates a chromatograph depicting the HPLC profile of the fraction (BV-3115). Whereas, FIG. 1B illustrates a table depicting the peak values of various pharmacologically active ingredients present in the fraction (BV-3115) obtained during the HPLC analysis of the fraction.

Various peaks corresponding to different pharmacologically active ingredients present in the fraction are shown in FIG. 1A. It was found that the peak corresponding to retention time 11.079 minute with the maximum area under the curve (1433542) was associated with Withaferin A. Whereas, the other smaller peaks were corresponding to the other pharmacologically active ingredients present in the fraction. Thus, it was concluded that the fraction contains Withaferin A in a concentration predominantly greater than the concentrations of all other pharmacologically active ingredients present in the fraction.

### Example 3

Hepato-cellular Carcinoma Cell Line (Hep G2 Cells)

Liver Hep G2 Cells were grown in Dulbeccos Modified Eagle Medium (DMEM) medium containing 5% fetal bovine serum and 2 mM L glutamine. Depending upon cell doubling time, between 5000 and 40000 cells were incubated into 96 well micro-titer plate with 100 .mu.l of DMEM per well. The plates were incubated at 37.degree. C., 5% CO.sub.2, 95% air, and 100% relative humidity for 24 hours prior to the addition of the experimental drug (BV-3115). After 24 hours of incubation, two plates of each cell line were fixed in-situ with Trichloroacetic Acid (TCA) as fixative agent to establish the cell population at the time of drug (BV-3115) addition. Prior to use, the experimental drug (BV-3115) was solubilized in dimethyl sulphoxide at 400 fold the desired final maximum test concentration and frozen. At the time of drug addition, an aliquot of frozen concentrate was thawed and diluted to twice the desired final maximum test concentration with complete medium containing 50 .mu.g/ml of gentamycin.

An additional four 10 fold or ½ log serial dilutions were made for a total of five drug concentrations (5 .mu.g/ml, 10 .mu.g/ml, 25 .mu.g/ml, 50 .mu.g/ml, and 100 .mu.g/ml) and a control. An aliquot of 100 .mu.l of each drug dilution was added to the appropriate well that already contained 100 .mu.l of DMEM. The plate was incubated at 37.degree. C., 5% CO.sub.2, 95% air, and 100% relative humidity. Cultures were removed from incubators into laminar flow hood. Fixative (50% TCA) was prepared by adding 22 ml of reagent grade water to TCA. The cells were fixed by gently layering ¼th volume of cold 50% TCA on top of the growth medium. The plates were incubated for one hour at 4.degree. C. and then rinse with water several times to remove TCA, serum proteins, etc. Plates were air dried and stored until use.

Blank background optical density was measured in wells incubated with growth medium without cells. SRB solution (0.4%) was added in an amount sufficient to cover the culture surface area (approx 50% of the culture medium volume). The cells were allowed to stain for 20 to 30 minutes. A wash solution was prepared by diluting the 10% acetic acid with 9 parts of water. At the end of the staining period, the stain was removed and the cells were rinsed quickly with 1% acetic acid. The process was repeated until unincorporated dye was removed.

After being rinsed, the culture was air dried until no moisture was visible. The incorporated dye was then solubilized in a culture medium volume. Tests performed in multi-well plates were read using an appropriate type of plate reader or the contents of individual wells may be transferred to appropriate size cuvets for spectrophotometer measurement. The different concentrations of BV-3115 (5 .mu.g/ml, 10 .mu.g/ml, 25 .mu.g/ml, 50 .mu.g/ml, and 100 .mu.g/ml) were used to determine the inhibition of Hep G2 Cells by BV-3115 using the SRB assay.

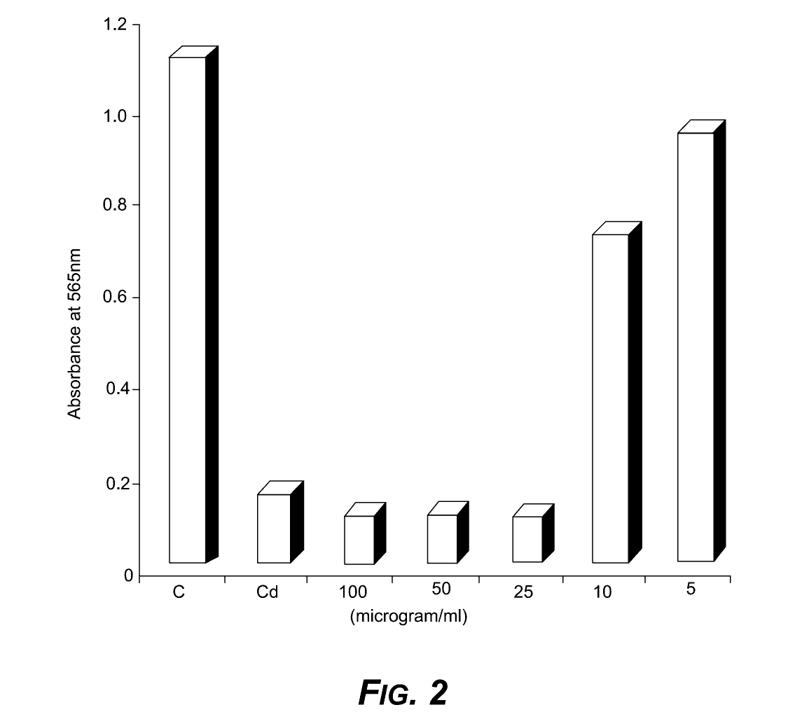

FIG. 2 illustrates effect of the fraction (BV-3115) on Hep G2 Cells using Sulforhodamine B (SRB) Assay. It was found that the fraction was effective in inhibiting liver hepatocellular carcinoma cell line at concentrations ranging from 25 .mu.g/ml to 100 .mu.g/ml.

### Example 4

Prostate Cancer Line (PC-3 Cells)

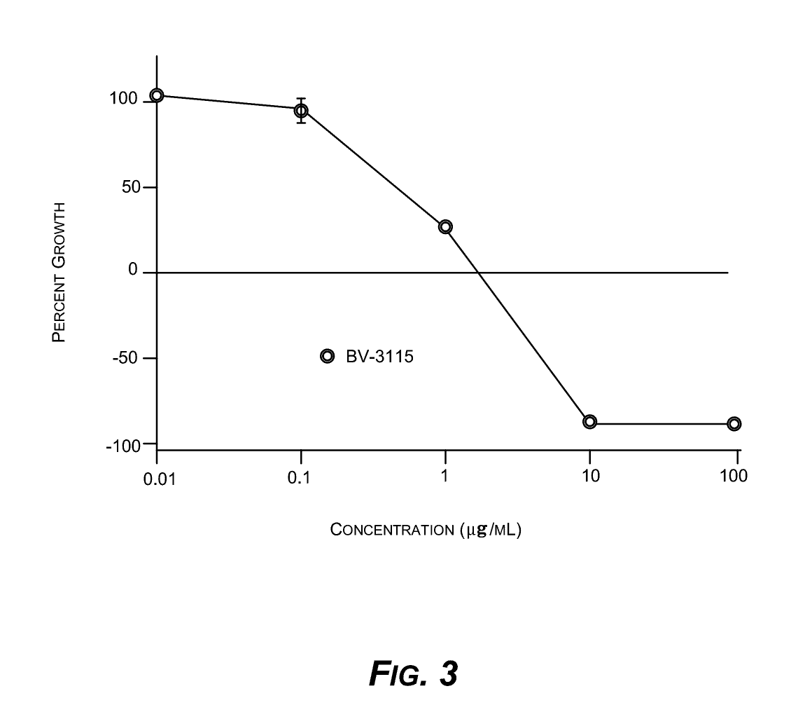

PC-3 Cells were suspended in growth medium, dispensed as 100 ml aliquots of 2.5.times.103 cells/well into 96-well micro-titer plate, and incubated at 37.degree. C., 5% CO.sub.2 for 24 hours. An additional 100 .mu.l of growth medium with 2 .mu.l of test solution, mitomycin or vehicle (40% DMSO), was added to each well followed by 72 hours incubation at 37.degree. C., and 5% CO.sub.2. The final concentration of DMSO in all wells was 0.4%. The test substance, BV-3115, was evaluated for its possible inhibitory effect on cell proliferation at concentrations of 0.01 .mu.g/mL, 0.1 .mu.g/mL, 1 .mu.g/mL, 10 .mu.g/mL and 100 .mu.g/mL in duplicate. At the end of incubation, 20 .mu.L of AlamarBlue reagent was added to each well for another 6 hours incubation before cell viability was determined by fluorescent intensity. Fluorescent intensity was measured using a GENios Plus micro-plate reader with excitation at 530 nm and emission at 590 nm. FIG. 3 illustrates effect of BV-3115 on inhibiting PC-3 Cells. It was found that BV-3115 is effective in inhibiting the proliferation of PC-3 cells. Further it was found that at concentrations ranging from 10 .mu.g/mL to 100 .mu.g/mL, BV-3115 shows optimum inhibition of the PC-3 cells.

### Example 5

Human Prostate Cancer Cell Line (DU-145 Cells)

Human Prostate Cancer (DU-145) Cells were grown in DMEM medium containing 5% fetal bovine serum and 2 mM L glutamine. Depending upon cell doubling time, between 5000 and 40000 cells were incubated into 96 well micro-titer plate with 100 .mu.l of DMEM per well. The plates were incubated at 37.degree. C., 5% CO.sub.2, 95% air, and 100% relative humidity for 24 hours prior to the addition of the experimental drug (BV-3115). After 24 hours of incubation, two plates of each cell line were fixed in-situ with TCA as fixative agent to establish the cell population at the time of drug (BV-3115) addition. Prior to use, the experimental drug (BV-3115) was solubilized in dimethyl sulphoxide at 400 fold the desired final maximum test concentration and frozen. At the time of drug addition, an aliquot of frozen concentrate was thawed and diluted to twice the desired final maximum test concentration with complete medium containing 50 .mu.g/ml of gentamycin.

An additional four 10 fold or ½ log serial dilutions were made for a total of five drug concentrations (5 .mu.g/ml, 10 .mu.g/ml, 25 .mu.g/ml, 50 .mu.g/ml, and 100 .mu.g/ml) and a control. An aliquot of 100 .mu.l of each drug dilution was added to the appropriate well that already contained 100 .mu.l of DMEM. The plate was incubated at 37.degree. C., 5% CO.sub.2, 95% air, and 100% relative humidity. Cultures were removed from incubators into laminar flow hood. Fixative (50% TCA) was prepared by adding 22 ml of reagent grade water to TCA. The cells were fixed by gently layering ¼th volume of cold 50% TCA on top of the growth medium. The plates were incubated for one hour at 4.degree. C. and then rinsed with water several times to remove TCA, serum proteins, etc. Plates were air dried and stored until use.

Blank background optical density was measured in wells incubated with growth medium without cells. SRB solution (0.4%) was added in an amount sufficient to cover the culture surface area (approx 50% of the culture medium volume). The cells were allowed to stain for 20 to 30 minutes. A wash solution was prepared by diluting the 10% acetic acid with 9 parts of water. At the end of the staining period, the stain was removed and the cells were rinsed quickly with 1% acetic acid. The process was repeated until unincorporated dye was removed.

After being rinsed, the culture was air dried until no moisture was visible. The incorporated dye was then solubilized in a culture medium volume. Tests performed in multi-well plates were read using an appropriate type of plate reader or the contents of individual wells may be transferred to appropriate size cuvets for spectrophotometer measurement. The different concentrations of BV-3115 (5 .mu.g/ml, 10 .mu.g/ml, 25 .mu.g/ml, 50 .mu.g/ml, and 100 .mu.g/ml) were used to determine the inhibition of DU-145 Cells by BV-3115 using the SRB assay.

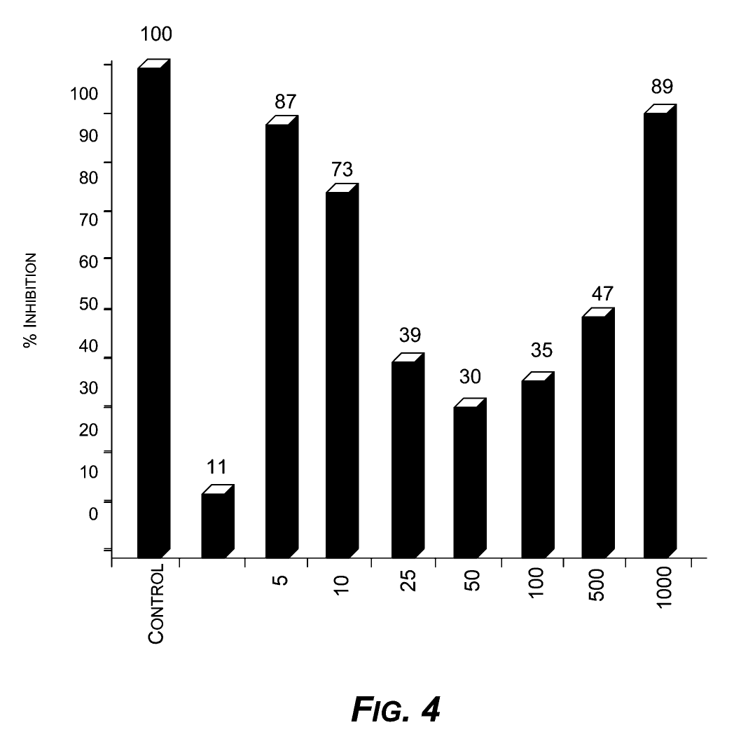

FIG. 4 illustrates effect of BV-3115 in inhibiting proliferation of DU-145 Cells, in-vitro. In-vitro screening, using SRB Assay, indicated that maximum inhibition in growth of cancerous cell line DU-145 was at concentrations between 25 .mu.g/ml and 100 .mu.g/ml. Further, the percentage inhibition in the growth of cancerous cell line DU-145 using BV-3115 was observed to be comparable with standard drug Adriamycin in SRB assay as described earlier.

### Example 6

Human Breast Cancer Cell Line (MCF-7 Cells)

Breast Cancer Cell Line (MCF-7 Cells) was grown in DMEM medium containing 5% fetal bovine serum and 2 mM L glutamine. Depending upon cell doubling time, between 5000 and 40000 cells were incubated into 96 well micro-titer plate with 100 .mu.l of DMEM per well. The plates were incubated at 37.degree. C., 5% CO.sub.2, 95% air, and 100% relative humidity for 24 hours prior to the addition of the experimental drug (BV-3115). After 24 hours of incubation, two plates of each cell line were fixed in-situ with TCA as fixative agent to establish the cell population at the time of drug (BV-3115) addition. Prior to use, the experimental drug (BV-3115) was solubilized in dimethyl sulphoxide at 400 fold the desired final maximum test concentration and frozen. At the time of drug addition, an aliquot of frozen concentrate was thawed and diluted to twice the desired final maximum test concentration with complete medium containing 50 .mu.g/ml of gentamycin.

An additional four 10 fold or ½ log serial dilutions were made for a total of five drug concentrations (5 .mu.g/ml, 10 .mu.g/ml, 25 .mu.g/ml, 50 .mu.g/ml, 100 .mu.g/ml and 1000 .mu.g/ml) and a control. An aliquot of 100 .mu.l of each drug dilution was added to the appropriate well that already contained 100 .mu.l of DMEM. The plate was incubated at 37.degree. C., 5% CO.sub.2, 95% air, and 100% relative humidity. Cultures were removed from incubators into laminar flow hood. Fixative (50% TCA) was prepared by adding 22 ml of reagent grade water to TCA. The cells were fixed by gently layering ¼th volume of cold 50% TCA on top of the growth medium. The plates were incubated for one hour at 4.degree. C. and then rinse with water several times to remove TCA, serum proteins, etc. Plates were air dried and stored until use.

Blank background optical density was measured in wells incubated with growth medium without cells. SRB solution (0.4%) was added in an amount sufficient to cover the culture surface area (approx 50% of the culture medium volume). The cells were allowed to stain for 20 to 30 minutes. A wash solution was prepared by diluting the 10% acetic acid with 9 parts of water. At the end of the staining period, the stain was removed and the cells were rinsed quickly with 1% acetic acid. The process was repeated until unincorporated dye was removed.

After being rinsed, the culture was air dried until no moisture was visible. The incorporated dye was then solubilized in a culture medium volume. Tests performed in multi-well plates were read using an appropriate type of plate reader or the contents of individual wells may be transferred to appropriate size cuvets for spectrophotometer measurement. The different concentrations of BV-3115 (5 .mu.g/ml, 10 .mu.g/ml, 25 .mu.g/ml, 50 .mu.g/ml, 100 .mu.g/ml and 1000 .mu.g/ml) were used to determine the inhibition of MCF-7 Cells by BV-3115 using the SRB assay.

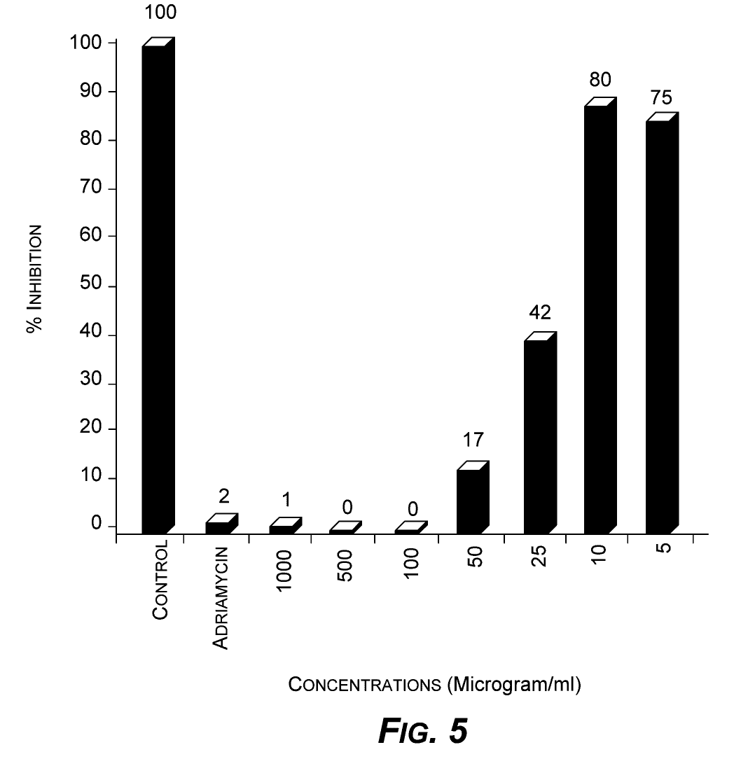

FIG. 5 illustrates effect of BV-3115 in inhibiting proliferation of MCF-7 Cells, in-vitro. In-vitro screening, using SRB Assay, indicates that maximum inhibition in growth of cancerous cell line MCF-7 was at concentrations between 100 .mu.g/ml and 1000 .mu.g/ml.

### Example 7

Human Colon Cancer Cell Line (Colo 320 DM Cells)

Human Colon Cancer (Colo 320 DM Cells) Cells were grown in Dulbeccos Modified Eagle Medium (DMEM) medium containing 5% fetal bovine serum and 2 mM L glutamine. Depending upon cell doubling time, between 5000 and 40000 cells were incubated into 96 well micro-titer plate with 100 .mu.l of DMEM per well. The plates were incubated at 37.degree. C., 5% CO.sub.2, 95% air, and 100% relative humidity for 24 hours prior to the addition of the experimental drug (BV-3115). After 24 hours of incubation, two plates of each cell line were fixed in-situ with TCA as fixative agent to establish the cell population at the time of drug (BV-3115) addition. Prior to use, the experimental drug (BV-3115) was solubilized in dimethyl sulphoxide at 400 fold the desired final maximum test concentration and frozen. At the time of drug addition, an aliquot of frozen concentrate was thawed and diluted to twice the desired final maximum test concentration with complete medium containing 50 .mu.g/ml of gentamycin.

An additional four 10 fold or ½log serial dilutions were made for a total of five drug concentrations (5 .mu.g/ml, 10 .mu.g/ml, 25 .mu.g/ml, 50 .mu.g/ml, 100 .mu.g/ml) and a control. An aliquot of 100 .mu.l of each drug dilution was added to the appropriate well that already contained 100 .mu.l of DMEM. The plate was incubated at 37.degree. C., 5% CO.sub.2, 95% air, and 100% relative humidity. Cultures were removed from incubators into laminar flow hood. Fixative (50% TCA) was prepared by adding 22 ml of reagent grade water to TCA. The cells were fixed by gently layering ¼th volume of cold 50% TCA on top of the growth medium. The plates were incubated for one hour at 4.degree. C. and then rinse with water several times to remove TCA, serum proteins, etc. Plates were air dried and stored until use.

Blank background optical density was measured in wells incubated with growth medium without cells. SRB solution (0.4%) was added in an amount sufficient to cover the culture surface area (approx 50% of the culture medium volume). The cells were allowed to stain for 20 to 30 minutes. A wash solution was prepared by diluting the 10% acetic acid with 9 parts of water. At the end of the staining period, the stain was removed and the cells were rinsed quickly with 1% acetic acid. The process was repeated until unincorporated dye was removed.

After being rinsed, the culture was air dried until no moisture was visible. The incorporated dye was then solubilized in a culture medium volume. Tests performed in multi-well plates were read using an appropriate type of plate reader or the contents of individual wells may be transferred to appropriate size cuvets for spectrophotometer measurement. The different concentrations of BV-3115 (5 .mu.g/ml, 10 .mu.g/ml, 25 .mu.g/ml, 50 .mu.g/ml, and 100 .mu.g/ml) were used to determine the inhibition of Colo 320 DM Cells by BV-3115 using the SRB assay.

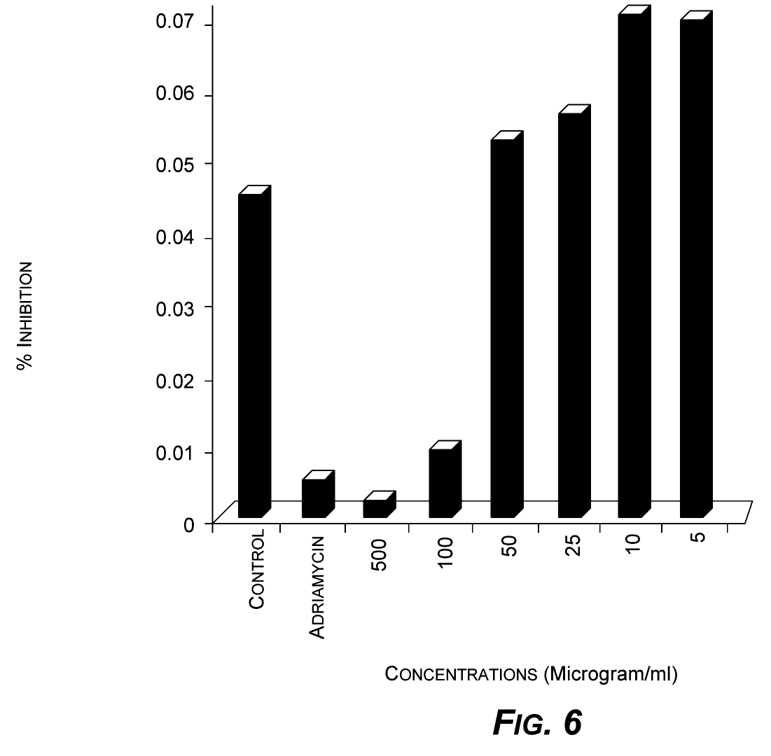

FIG. 6 illustrates effect of BV-3115 in inhibiting proliferation of Colo 320 DM Cells, in-vitro. In-vitro screening, using SRB Assay, indicates that maximum inhibition in growth of cancerous cell line Colo 320 DM was at concentrations between 50 .mu.g/ml and 500 .mu.g/ml. Further, the percentage inhibition in the growth of above mentioned cancerous cells of Colo 320 DM Cell Line with BV-3115 was found to be comparable with standard drug Adriamycin.

### Example 8

In-Vivo efficacy in Sarcoma Model in Mice

Healthy adult male mice of BALB/c strain (hereinafter, “animals”) weighing between 18 to 22 g were selected randomly. The animals were divided in to six groups. Each animal was identified by color marking. Each cage was identified by a label according to the group. The label contained the name of experiment, number of mice in that cage, dose (mg/kg) and date of initiation and completion of experiment. The groups received treatments for a period of 15 days. On Day 16, samples of blood were withdrawn, under anesthesia, from orbital sinus of the animals. The samples were collected in tubes containing Heparin as an anticoagulant.

Group I was treated as vehicle control and received vehicle (Corn oil) orally, in equal volume as in treatment groups. Group II received only tumor cells (1.times.10.sup.6 cells) intra-peritoneally. Group III received tumor cells (1.times.10.sup.6 cells) and cyclophosphamide (150 mg/kg body weight) intra-peritoneally. Group IV and Group V received Tumor cells and BV-3115 at 100 mg/kg body weight and 250 mg/kg body weight respectively. Group VI received Tumor cells, cyclophosphamide and BV-3115 at 100 mg/kg body weight. No mortality was observed in any of the groups.

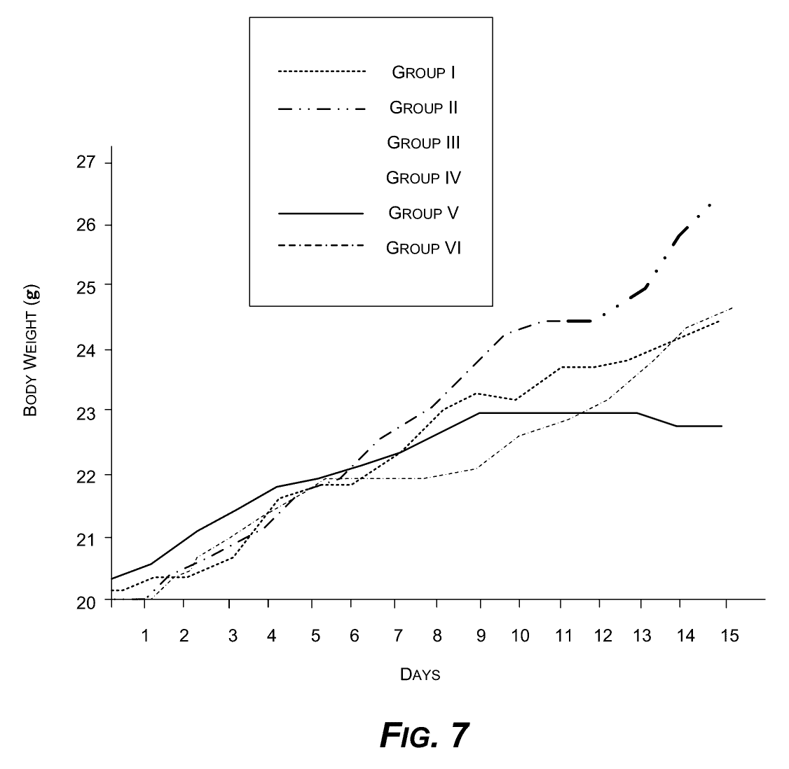

Slight excitation was observed in three animals of Group V (BV-3115 at 250 mg/kg body weight). No clinically abnormal signs were observed in any other groups. FIG. 7 illustrates body weight gain/loss in the animals used in Sarcoma Model as compared with the control group. Normal body weight gain was observed in Group I animals. Group II animals showed statistically significant increase in body weight when compared with Group I (P<0.05). Group III animals showed normal weight gain till Day 10, thereafter sudden decrease in weights was observed when compared to Group I (P<0.05). The Groups IV and VI animals showed normal body weight gain throughout the experiment when compared to control (P>0.05). The Group V animals showed statistically significant decrease in body weights when compared to Group I (P<0.05). All the treatment groups (III, IV, V and VI) showed significant decrease in body weight when compared to Group II.

Hematology Parameters

Significant decrease in hemoglobin was observed in Group II as compared to group I (P<0.05). In all the treatment groups the hemoglobin content increased in varying degrees. The hemoglobin values of group V and VI were found to be comparable to group I (P>0.05). Significant decrease in RBC count was observed in Group II as compared to group I (P<0.05). In all the treatment groups the RBC count increased in varying degrees. The RBC values of group V were found to be comparable to group I (P>0.05) Group II animals showed very high WBC count (P<0.05), compared with Group I). While there was statistically significant decrease in white blood cells in group III and VI animals when compared with Group II (P<0.05). The values of white blood cells in group IV and V were found to be comparable to those of Group I. The groups II animals showed significant increase in platelet count (P<0.05, compared with Group I). The values of platelets in all other groups were found to be comparable to Group I (vehicle control).

Based on the above findings of the Sarcoma Model in mice it was concluded that that BV-3115 reduces the Tumor growth compared to control animals.

### Example 9

Sub-Chronic Toxicity in Rats Treated With BV-3115 for 28 Days

Albino rats (hereinafter, “animals”) of Sprague Dawley strain weighing between 60 to 80 g were selected randomly. Each animal was identified by colour marking. Then animals were divided into five dose groups randomly. Each cage was identified by a label according to the group. The label contained name of experiment, number of rats in that cage, dose (mg/kg) and date of initiation and completion of experiment.

Group I received control vehicle (edible oil) for 28 days, whereas Group II, Group III, Group IV and Group V received levels of 100 mg/kg body weight, 200 mg/kg body weight, 400 mg/kg body weight and 800 mg/kg body weight of BV-3115, respectively. Each animal received the treatment for 28 days. The drug was administered orally as a suspension in edible oil. The concentration of suspension was prepared to give a constant dosage volume of 10 ml/kg body weight. The control animals received vehicle alone at the same dosage volume. The dosing formulations were prepared daily. The dosage volume to individual animals was adjusted according to the most recently recorded body weights. The treatment in this manner was continued once a day for a period of 28 days.

Throughout the study, all animals were checked twice daily for dead or moribund animals. Signs noted included, but not be limited to, changes in skin, fur, eyes, and mucous membranes, occurrence of secretions and excretions and autonomic activity (e.g. lacrimation, piloerection, pupil size, and unusual respiratory pattern). Changes in gait, posture and response to handling as well as the presence of clonic or tonic movements, stereotypes (e.g. excessive grooming, repetitive circling) or bizarre behavior (e.g. self mutilation, walking backwards) were also recorded. All signs of ill-health, behavioral changes or reaction to the treatment were noted for individual animals and the circumstances of any death were recorded. Dated and signed records of appearance change and disappearance of clinical signs was maintained on clinical history record sheet for each animal.

The body weight of each animal was recorded prior to commencement of the treatment, on the day of commencement of the treatment and weekly thereafter. In addition the body weights were also taken at the time of necropsy. The test was tested for dose range finding study by administering the drug orally daily for 28 days at the dose levels of 100 mg/kg body weight, 200 mg/kg body weight, 400 mg/kg body weight and 800 mg/kg body weight.

Decrease in body weights and food consumption in Group IV and V was observed during the test period. Increase in alkaline phosphatase levels with decrease in hemoglobin, PCV and RBCs was also observed. Increase in liver weight in all animals of Group IV and V and decrease in testicular size of male rats in Group V (800 mg/kg body weight). Mortality in female rats was also observed in Group V (800 mg/kg body weight). On the basis of the findings, it was concluded that BV-3115 does not have any significant toxic effects in rats at doses of 100 mg/kg and 200 mg/kg body weight, and appears to exhibit some toxic effects in rats at doses of 400 mg/kg body weight and 800 mg/kg body weight.

### Example 10

Anti-Inflammatory Effect of BV-3115

Edema represents an early phase of inflammation characterized by an initial release of histamine and 5-hydroxytryptamine (5-HT) producing an early vascular permeability followed by release of cytokines, further contributing to increased vascular permeability. Lastly, the prostaglandins and slow releasing substances (SRS) are released to maintain the increased vascular permeability produced by histamine, 5-HT and cytokines This model was used for studying anti-inflammatory activity of the fraction.

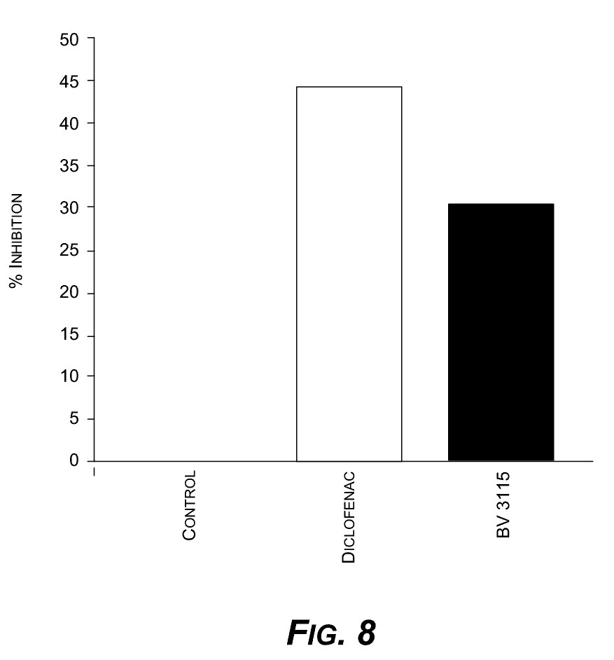

A standard solution of Carrageenan, an inflammatory agent was injected in the rat paw to produce swelling which was measured by a Plethysmograph. This instrument measures the extent of paw swelling due to injection of Carrageenan. A comparative study of extent of rat paw swelling with and without drug was conducted using this instrument. The effect of BV-3115 and another known NSAID, Diclofenac were used in this study. FIG. 8 illustrates percent inhibition of rat paw edema using BV-3115 as compared with Diclofenac and control. The fraction (BV-3115) was found to exhibit a significant anti-inflammatory effect as compared with Diclofenac.

Various embodiments of the present invention provide method for obtaining one or more fractions from WS.

The one or more fractions contain Withaferin A in a concentration greater than concentrations of other pharmacologically active ingredients present in the one or more fractions.

The one or more fractions obtained in accordance with the various embodiments of the invention are less toxic in mammals as compared to pure Withaferin A. Further, the invention provides one or more fractions and one or more compositions containing the one or more fractions that are effective in various mammalian cancers. In addition the invention provides one or more fractions and one or more compositions that when used in combination with chemotherapy and/or radiotherapy reduce one or more side-effects and toxicity otherwise associated with the chemotherapy and/or radiotherapy.

Ashwagandha (Withania somnifera Dunal., Solanaceae) is one of the most reputed medicinal plants of Ayurveda, the traditional medical system. Several of its traditionally proclaimed medicinal properties have been corroborated by recent molecular pharmacological investigations and have been shown to be associated with its specific secondary metabolites known as withanolides, the novel group of ergostane skeletal phytosteroids named after the plant. Withanolides are structurally distinct from tropane/nortropane alkaloids (usually found in Solanaceae plants) and are produced only by a few genera within Solanaceae. W. somnifera contains many structurally diverse withanolides in its leaves as well as roots.

A total of 62 major and minor primary and secondary metabolites from leaves and 48 from roots have been identified. 29 of these are common to the two tissues. These included fatty acids, organic acids, amino acids, sugars and sterol based compounds. 11 bioactive sterol-lactone molecules have also been identified. 27 of the identified metabolites have been quantified. Highly significant qualitative and quantitative differences are noticed between the leaf and root tissues, particularly with respect to the secondary metabolites.

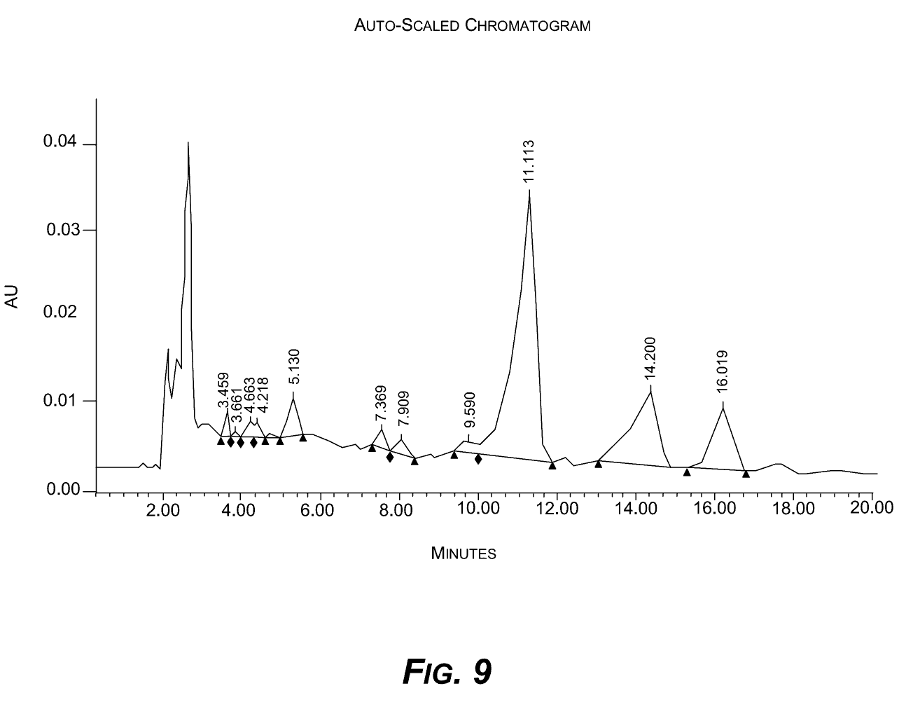

FIG. 9 shows major and minor HPLC peaks for an example BV-3115 adjunct compound. Ten different peaks are evident. Out of the ten different retention times, retention time of 11.11 minutes was matched with Withaferine A along with other minor peaks at different minutes like 14.20 min and 16.019 min.

In an example LC-MS/MS estimation of BV-3115, results showed the different number of retention times of the components along with the standard WA matched well with the retention time of components such as Withanolide A, 24,25,dihydroxy Withanolide D, 27 Deoxy Withaferine A and Somniferinein standards. In the LC/MS studies, the quantity of each component present was estimated by comparing with AUC of the respective std component. In the LCMS analysis of an example analysis of BV-3115, presence of 10-12 different mass values was observed. Out of the above ten different mass values, the molecule depicting the mass values were identified.

From the above studies it was concluded that BV-3115 is not a naturally occurring fraction, and contains non-naturally occurring species. A novel and unique fraction, for example, contains at least a combination of Withanolide-A; 24,25,dihydroxy Withanolide D5-B; 6B Epoxy-4Bhydroxy-1-oxo 20S; 22Rwitha 2,24dienolide i.e 27 Deoxy Withaferine A and Somniferinein.

In non cancerous cells, like mouse macrophage RAW264.7, the example compounds do not block any normal non-cancerous ribosomal functions even at 100 ug/ml dose (direct binding to rRNAs and elongation step in translational machine) as detected by total cellular proteins by Sulforhodamine B colorimetric assay. But in a significantly lesser dose, in cancerous cells, by the same assay, the example compounds cause a loss of signal. This provides that the novel mixture/formulations of the example BV-3115 compounds block the cellular proliferations by a distinct mechanism, rather just blocking the translational machinery of the cell.

The example BV-3115 compounds prevent cyclophosphamide related toxicity/and body weight loss. In combination with cyclophosphamide, the example compounds protect against severe side effects of this chemotherapy drug in animal models (protects against loss of body weight in animals with the cancers). Cyclophosphamide is a pro-drug. With the help of the cytochrome system in the liver, the agent is biotransformed into phosphoramide mustard and acrolein, which are very active compounds. Phosphoramide mustard has an ability to introduce alkyl radicals into DNA strands with interferes DNA replication by DNA cross-linkage. Cross-linked DNA in cancer cell is unable to complete normal cell division. Thus, it stops cancer cells from growing, causing them to die. Cyclophosphamide also produces immunosuppressive effects possibly through a cytotoxic effect on lymphocytes and causes severe body weight loss in animals or patients. The results show that the example BV-3115 compounds alleviate the body weight loss due to cancer.

Mouse Sarcoma Model:

A group of male mice approximately 7-8 weeks old and weighing between 18-22 gm were used for this study. Animal room temperature & relative humidity were maintained at 22-30 C. and 30-70% respectively. All mice were given free access to purified water (Purified with Aquaguard) & standard pelleted laboratory animal feed. After acclimation period of 7 days, each animal was weighed and then the animals were randomly selected according to body weights and allocated to the different treatment groups, so that mean body weights of groups were not statistically different before starting the experiment. Tumor implantation with S-180 cells 1×106 cells I.P. to all groups except group I (sham control no tumor, no drug). On day 15 because of tumor it weighed 45% more than control, where as a known drug reduced the body weight alarmingly lower than sham control because of its toxicity. Surprisingly the example BV-3115 compounds do better job in reducing the tumor dependent body weight gain but also reduce the side effects of toxic cyclophosphamide. This shows that the example BV-3115 formulations have properties to counteract the toxic effects of Cyclophophamide.

BV-3115 does not block ribosome function in normal cells. In a mouse macrophage study, RAW 264.7 cell line (ATCC TIB-71) and human breast cancer (MCF-7) cells were incubated in an atmosphere of 5% CO2 at 37 degrees C. with either DMSO or four different doses of BV-3115 for 24 hrs and total cellar protein content was checked by SRB assay. Both cells were plated with a seeding density of 5000 cells/well. The culture medium was DMEM supplemented with 3% fetal bovine serum and 1% Antibiotic-Antimycotic. As SRB assay measures total protein content (ribosomal function), Novel Formulation affects differently in metastatic cancer cells vs normal cell growth as shown in the following table. Raw264.7 cells, suppose to be normal barely affects the protein content even at 100 ug/ml. This clearly shows the mechanism of vimentin related directly binding by just Withaferin A is not the same for BV-3115.

Those skilled in the art will realize that the above-recognized advantages and other advantages described herein are merely exemplary and are not meant to be a complete rendering of all of the advantages of the various embodiments of the present invention.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The present invention is defined solely by the appended claims including any amendments made during the pendency of this application and all equivalents of those claims as issued.

1-20. (canceled)

21. A method of making an adjunct to potentiate blocking of ribosomal functions in tumor cells while preventing weight loss of a patient during cyclophosphamide chemotherapy, comprising:
obtaining a plant material of Withania somnifera;
subjecting the plant material to hydro-alcoholic extraction in presence of a water-insoluble solvent to obtain an extract;
isolating from the extract substantially all of each of the components:
withaferin A,
withanolide A,
24,25-dihydroxy withanolide D,
5B,6B-epoxy-4B-hydroxy-1-oxo-20S,22R-witha-2,24-dienolide, and
somniferinin; and
mixing the isolated components together in an oral preparation.

22. The method of claim 21, wherein the withaferin A has a molecular weight of 471 and a chemical formula of C₂₈H₃₈O₆, the withanolide A has a molecular weight of 471 and a chemical formula of C₂₈H₃₈O₆, the 24,25-dihydroxy withanolide D has a molecular weight of 488.491 and a chemical formula of C₂₈H₄₀O₇, the 5B,6B-epoxy-4B-hydroxy-1-oxo-20S,22R-witha-2,24-dienolide has a molecular weight of 455.466 and a chemical formula of C₂₈H₃₈O₇, and the somniferinin has a molecular weight of 489.49 and a chemical formula of C₂₈H₄₂O₇.

23. The method of claim 21, further comprising reacting one or more of the isolated components for depigmenting, defatting, or detoxifying one or more of the isolated components.

24. A method of preventing weight loss of a patient during cyclophosphamide chemotherapy, comprising:
isolating components
withaferin A,
withanolide A,
24,25-dihydroxy withanolide D,
5B,6B-epoxy-4B-hydroxy-1-oxo-20S,22R-witha-2,24-dienolide, and
somniferinin
from a Withania somnifera plant matter;
mixing the isolated components together in an oral preparation; and
administering the oral preparation to the patient at a dosage in a range of approximately 100-200 mg/kg of body weight as an adjunct to an administration of the cyclophosphamide chemotherapy to the patient.

25. The method of claim 24, further comprising reacting one or more of the isolated components for depigmenting, defatting, or detoxifying one or more of the isolated components.
